(19) United States
(12) Patent Application Publication
(10) Pub. No.: US 2026/0083897 A1
(43) Pub. Date: Mar. 26, 2026

(54) DRESSING FOR USE IN THE NEGATIVE PRESSURE THERAPY OF WOUNDS, NEGATIVE PRESSURE WOUND THERAPY KIT, AND NEGATIVE PRESSURE WOUND THERAPY SYSTEM

(71) Applicant: PAUL HARTMANN AG, Heidenheim (DE)

(72) Inventor: Martin DEIBLER, Herbrechtingen (DE)

(73) Assignee: PAUL HARTMANN AG, Heidenheim (DE)

(21) Appl. No.: 19/140,222

(22) PCT Filed: Dec. 6, 2023
(86) PCT No.: PCT/EP2023/084515

§ 371 Date: Jun. 17, 2025

(30) Foreign Application Priority Data
Dec. 19, 2022 (DE) 10 2022 133 930.0

(51) Int. Cl. A61M 1/00; A61F 13/00; A61F 13/0206; A61F 13/05

(52) U.S. Cl. CPC A61M 1/912; A61F 13/00076; A61F 13/0206; A61F 13/05; A61M 1/915; A61M 2205/75; A61M 2209/06

(57) ABSTRACT

The invention relates to a dressing (16) for use in the negative pressure therapy of wounds, having: a wound contact layer (28); an air-impermeable top layer (34), wherein a receiving space (36) is formed between the top layer and the wound contact layer, wherein the wound contact layer is designed such that it is liquid-permeable and draws wound exudate from the wound into the receiving space, and wherein a connection opening (26) is formed in the top layer; and at least one distribution layer (50) arranged in the receiving space, wherein the distribution layer extends in two surface directions (X, Y) and is designed to distribute wound exudate in the surface directions. According to the invention, the distribution layer is formed by a flat material web portion (52), a first side (56) of the flat material web portion is designed as a structure defined by elevations (60) that are formed integrally with a plane (54) of the flat material web portion, a terminal through-opening (64) is formed in at least some of the elevations, and a cohesive intermediate space (66) is formed between the elevations.

The present invention relates to a wound dressing for use in the negative pressure wound therapy. The present invention also relates to a negative pressure wound therapy kit comprising such a wound dressing. The present invention further relates to a negative pressure wound therapy system comprising a negative pressure source and such a negative pressure wound therapy kit.

Negative pressure wound therapy (NPWT) is an innovative method of treating wounds with a wide range of indications. These include, for example, acute skin and soft tissue defects, wound healing disorders, chronic wounds etc. The aim of negative pressure therapy is to stimulate the growth of granulation tissue and to promote the healing process. In the context of negative pressure therapy, this aim is pursued by establishing a negative pressure in the region of a wound, thereby enabling effective draining of wound exudate from the wound.

Wound dressings used in negative pressure therapy typically include a wound contact layer for application to the wound bed of a wound and an air-impermeable cover layer for airtight closure of the wound. A receiving space is formed between the wound contact layer and the cover layer. The wound contact layer is in liquid-permeable form for discharge of wound exudate from the wound into the receiving space. The cover layer comprises a connection opening for negative pressure-tight fluidic connection of the receiving space to a negative pressure source. When the wound dressing is applied to a wound as intended and the negative pressure source has been fluidically connected to the connection opening in a negative pressure-tight manner, the negative pressure source can establish a negative pressure in the wound space and the receiving space. In known wound dressings, at least one distributor layer is generally disposed in the receiving space, and extends in two spatial directions and is designed to distribute wound exudate in the spatial directions. A distribution in the spatial directions is also referred to hereinafter as lateral distribution. Lateral distribution of the wound exudate prevents accumulation of wound exudate and any associated blockages of constituents of the wound dressing. In addition, the distributor layer also promotes lateral distribution of the negative pressure in the wound cavity.

A generic wound dressing is known, for example, from patents EP 3 287 106 B1 and EP 2 879 636 B1. These patent specifications propose various materials for the distributor layer, for example an open-cell foam, a spacer fabric or a nonwoven fabric. Such materials do meet the fundamental requirements on a distributor layer. However, the use of the materials is comparatively costly. This was consciously accepted in the case of previously known wound dressings.

It is an object of the invention, in a wound dressing for negative pressure wound therapy, to provide a way of using inexpensive means to assure effective lateral distribution of wound exudate in the receiving space.

The object is achieved in accordance with the invention by a wound dressing as claimed in claim 1, by a negative pressure wound therapy kit as claimed in claim 22, and by a negative pressure wound therapy system as claimed in claim 23.

The dependent claims and the description indicate advantageous variants and embodiments.

The invention thus provides a wound dressing for use in negative pressure wound therapy. The wound dressing comprises a wound contact layer for application to the wound bed of a wound. The wound contact layer comprises a smooth first side and a smooth second side facing away from the first side. In addition, the wound dressing of the invention comprises an air-impermeable cover layer for airtight closure of the wound.

A receiving space is formed between the cover layer and the wound contact layer. The wound contact layer is in liquid-permeable form for discharge of wound exudate from the wound into the receiving space. The wound contact layer is preferably in perforated form, such that the wound exudate can pass through the wound contact layer via perforations or via passage openings formed in the wound contact layer. However, the wound contact layer may also be intrinsically liquid-permeable. A connection opening for negative pressure-tight fluidic connection of the receiving space to a negative pressure source is formed in the cover layer. The wound dressing of the invention further comprises at least one distributor layer disposed in the receiving space. The distributor layer extends in two spatial directions and is designed to distribute wound exudate in the spatial directions.

What is now envisaged is that the distributor layer is formed by a flat material web section, that a first side of the flat material web section is structured by elevations integrally with a plane of the flat material web section, that an end-face passage opening is formed in at least some of the elevations, in particular in all elevations, and that a contiguous interspace is formed between the elevations such that wound exudate is distributable in the spatial directions in the interspace.

It has been found that, surprisingly, a flat material web section as described above is particularly suitable as a distributor layer or as a component of a distributor layer in view of its properties. By virtue of the end-face passage openings, wound exudate drained into the receiving space can be transported through the flat material web section orthogonally to the spatial directions. In the interspace or through the interspace, wound exudate can be distributed in the spatial directions, in particular along the first side of the flat material web section. A flat material web section of the above design is obtainable inexpensively. This results in particular from the fact that suitable elevations can be formed in a flat material web or a flat material web section without taking much time. The use of a flat material web section in a distributor layer or as a distributor layer also has the advantage that it is possible to obtain a wound dressing that assures high patient comfort. This follows in particular from the fact that a wound dressing can be implemented with a low construction height.

A flat material web section is a section of a flat material web. For example, the flat material web section is cut out of a flat material web. The elevations are preferably already formed in the flat material web. However, it is also possible for the elevations to be formed only in the flat material web section. In particular, the flat material web is a polymer film web, and so the flat material web section takes the form of a polymer film.

Preferably, the flat material web section on the first side has an elevation density of at least 50 elevations per cm2, preferably an elevation density of at least 100 elevations per cm2, more preferably an elevation density of at least 200 elevations per cm2. Such high elevation densities are associated with elevations of small dimensions. The stability of the flat material web section is then impaired slightly at most by the elevations and the end-face passage openings. On the other hand, limitation of the elevation density is also advantageous in order to achieve sufficiently large distances between adjacent elevations. Preferably, the flat material web section on the first side has an elevation density of not more than 1000 elevations per cm2, more preferably an elevation density of not more than 500 elevations per cm2. More preferably, the flat material web section has an elevation density of at least 200 elevations per cm2 to at most 400 elevations per cm2. Preferably, the diameter of the end-face passage openings is at least 100 μm, preferably at least 200 μm. Preferably, the diameter of the end-face passage openings is not more than 1000 μm, preferably not more than 500 μm. More preferably, the diameter of the end-face passage openings is at least 200 μm and at most 500 μm.

What is meant here by “airtight closure” is that, taking into account the negative pressure source used, the negative pressure needed for negative pressure wound therapy can be maintained. It is therefore also possible to use cover layers having slight gas permeability, provided that the negative pressure needed for negative pressure therapy can be maintained.

The same applies to the understanding of the term “negative pressure-tight fluid connection”. What is meant here by a “negative pressure-tight fluidic connection” is that, taking account of the negative pressure source used, the negative pressure needed for negative pressure wound therapy can be maintained in the fluidically interconnected cavities.

In the context of negative pressure therapy, a pressure differential between the air pressure within the negative pressure wound dressing and the ambient air pressure of at least 20 mm Hg (millimeters of mercury) to a maximum of 250 mm Hg is typically established. 1 mm Hg corresponds to one torr or 133.322 Pa (pascals).

A “smooth side” of a layer is not supposed to mean here that the smooth side is necessarily free of any unevenness. Instead, a “smooth side” refers to a side that is smooth compared to the structured first side of the flat material web section. Accordingly, there may be unevenness even on a smooth side of a layer, for example as a result of irregularities in the material thickness of the layer in question. However, any unevenness is of a smaller order of magnitude in terms of height than the elevations of the structured first side of the flat material web section.

In a preferred embodiment, the elevations are conical, cylindrical, frustopyramidal or hyperboloid. Such elevations can be introduced in a procedurally simple manner into the flat material web or the flat material web section. In addition, a highly branched interspace is created, which is accompanied by effective distribution of wound exudate in the spatial directions. The elevations are preferably isolated from one another in insular form. The elevations preferably have a height of 200 μm to 1000 μm.

In a preferred embodiment, at least in a central region of the flat material web section, each of the elevations is surrounded by three to eight further elevations, where the further elevations together form a regular polygon. An interspace thus created assures particularly effective distribution of wound exudate. The further elevations more preferably form a regular hexagon.

Preferably, a second side of the flat material web section facing away from the structured first side is smooth. It has been found that the elevations and the interspace formed in between on the first side of the flat material web section are sufficient for effective distribution of wound exudate in the spatial directions. A flat material web section with a structured first side and a smooth second side is easy to implement in terms of manufacturing.

The elevations are preferably manufactured by vacuum thermoforming. Such formation of the elevations can be integrated into the production of the flat material web or of the flat material web section in a time-saving manner. For this purpose, for example, a still-molten extruded or calendered flat material web, in particular polymer film web, is passed across a rotating perforated roll under reduced pressure. The flat material web is then pulled region by region into holes of the perforated roll under reduced pressure by a negative pressure in the perforated roll under reduced pressure, which forms the elevations. The shape of the elevations obtained is determined, for example, by the outline of the holes and by the negative pressure used. Preferably, the negative pressure in the perforated roll under reduced pressure is adjusted such that the end faces of at least some of the elevations open up. The elevations and the passage openings are then formed in the same process step.

In a preferred embodiment, the flat material web section has been made from polyethylene, polyurethane, polyvinylchloride or mixtures thereof. Because of their properties, these plastics are particularly suitable for the formation of the flat material web section. For instance, the plastics are sufficiently flexible that a wound dressing with advantageous drapability can be obtained. Furthermore, it is possible to achieve a sufficiently rigid flat material web section, such that it is assured that the contiguous interspace is maintained even when a negative pressure has been established.

In a preferred embodiment, the distributor layer is disposed in the receiving space such that the elevations protrude in the direction of the cover layer. In the elevations, the wound exudate is transported by capillary forces in the direction of the end-face passage openings. If the elevations extend away from the lower ply, the capillary forces accordingly support the transport of wound exudate through the flat material web section.

In a preferred embodiment, at least one further distributor layer disposed in the receiving space extends in two spatial directions and is designed to distribute wound exudate in the spatial directions, the further distributor layer is formed by a flat material web section, a first side of the flat material web section is structured by elevations integrally with a plane of the flat material web section, an end-face passage opening is formed in at least some of the elevations, in particular in all elevations, a contiguous interspace is formed between the elevations such that wound exudate is distributable in the spatial directions in the interspace, and the distributor layers are stacked one on top of the other. If two layers are stacked one on top of the other, the sides of the two layers extend parallel to each other. The layers may be stacked directly one on top of the other. However, it is also possible for at least one further layer to be disposed between the two layers. The additional distributor layer can increase the capacity of the wound dressing for wound exudate. Further configuration of the further distributor layer can be accomplished using the features described in association with the distributor layer. The distributor layer and the further distributor layer are preferably the same. However, the distributor layer and the further distributor layer may also differ from each other in at least one feature, for example in the material composition and/or the shape of the elevations.

Preferably, the distributor layer and the further distributor layer are arranged such that the elevations of the distributor layers protrude in the same direction. Such an arrangement achieves the effect that the aforementioned capillary forces support the transport of wound exudate in the same direction. Preferably, the distributor layer and the further distributor layer are arranged such that the elevations protrude in the direction of the cover layer. If the distributor layer and the further distributor layer are stacked directly one on top of the other, the advantage additionally arises that the interspace between the elevations of one distributor layer is not blocked by the elevations of the other distributor layer. Such blockage of the interspace could otherwise at least complicate distribution of the wound exudate and/or the negative pressure in the spatial directions.

In a preferred embodiment, at least one absorption layer disposed in the receiving space is designed to absorb wound exudate and store absorbed wound exudate. The absorption layer increases the capacity of the wound dressing for wound exudate. Preferably, the absorption layer comprises a hydrophilic material for receiving and storing of wound exudate. Suitable hydrophilic materials include, for example, superabsorbent polymers, superabsorbent fibers, cellulose and/or a hydrophilic nonwoven material. The presence of the absorption layer is preferred. However, the disclosure also covers wound dressings in which no absorption layer is disposed in the receiving space.

Preferably, the distributor layer or at least one of the distributor layers is disposed between the absorption layer and the wound contact layer. As a result, wound exudate is distributed in the spatial directions by the distributor layer before being absorbed into the absorption layer. This can increase the absorption efficiency of the absorption layer because a greater surface area of the absorption layer is utilized for the absorption of wound exudate. In addition, the distribution of wound exudate in the spatial directions can prevent blockage of the absorption layer. Such blockage could result from concentrated, undistributed accumulations of wound exudate in the absorption layer.

Preferably, the distributor layer or at least one of the distributor layers is disposed between the absorption layer and the cover layer. This prevents blockage of the absorption layer on its side facing the cover layer. The distributor layer disposed between the absorption layer and the cover layer also prevents individual areas of the absorption layer from being isolated from the negative pressure. Such isolation of individual regions could impair the efficiency of the wound dressing with regard to the draining of wound exudate from the wound.

In a preferred embodiment, the absorption layer is in direct contact with the distributor layer and/or the further distributor layer. Direct contact can ensure effective passage of wound exudate from one of the layers to the other of the layers.

In a preferred embodiment, at least one further absorption layer disposed in the receiving space is designed to absorb wound exudate and store absorbed wound exudate, and the absorption layer and the further absorption layer are stacked one on top of the other. The further absorption layer can increase the amount of absorbent materials present. Compared with only one absorption layer of correspondingly larger dimensions, the advantage is that the surface area available for absorption is increased. Further configuration of the further absorption layer can be accomplished using the features described in association with the absorption layer. The absorption layer and the further absorption layer are preferably the same. However, the absorption layer and the further absorption layer may also differ from each other in at least one feature, for example in layer thickness and/or material composition.

Preferably, the distributor layer or at least one of the distributor layers is disposed between the absorption layer and the further absorption layer. This can increase the absorption efficiency of the absorption layer closer to the cover layer. The reason for this is that, because of the lateral distribution of wound exudate between the two absorption layers, a larger surface area of the absorption layer closer to the cover layer is utilized for the absorption of wound exudate.

There follows an enumeration of various preferred embodiments of the wound dressing that differ from one another in the specific combination of layers disposed in the receiving space. In a first particularly preferred embodiment of the wound dressing, only one distributor layer is disposed in the receiving space. There is no further distributor layer or absorption layer. In a second particularly preferred embodiment of the wound dressing, only a distributor layer and a further distributor layer are disposed in the receiving space. In a third particularly preferred embodiment of the wound dressing, only one distributor layer and one absorption layer are disposed in the receiving space. The distributor layer is disposed between the absorption layer and the wound contact layer or between the absorption layer and the cover layer. In a fourth particularly preferred embodiment of the wound dressing, only a distributor layer, a further distributor layer and an absorption layer are disposed in the receiving space. The distributor layer and the further distributor layer are each disposed on opposite sides of the absorption layer. In a fifth particularly preferred embodiment of the wound dressing, an absorption layer and a further absorption layer are disposed in the receiving space. A distributor layer is disposed between the two absorption layers. A further distributor layer is disposed on a side of the absorption layer that faces away from the further absorption layer. A further distributor layer is disposed on a side of the further absorption layer that faces away from the absorption layer.

In a preferred embodiment, the cover layer comprises a central region and an edge region enclosing the central region, the wound contact layer comprises a central region and an edge region enclosing the central region, and the edge region of the cover layer is bonded to the edge region of the wound contact layer such that the receiving space is formed solely between the central region of the cover layer and the central region of the wound contact layer. As a result, the layers disposed in the receiving space are framed in the lateral direction. By virtue of the bonding of the edge region of the wound contact layer to the edge region of the cover layer, the receiving space is formed effectively in the form of a closed pocket. The bonding of the edge regions to one another has the advantage that it is possible to dispense with any connection of the layers disposed in the receiving space to the wound contact layer and/or the cover layer, and with any connection of the layers disposed in the receiving space to one another. This means that, in the production of the wound dressing, it is possible to dispense with process steps involving the connection of layers, resulting in cost savings.

The edge region of the cover layer and the edge region of the wound contact layer are preferably bonded to one another by an adhesive bond or by a weld bond. The bond may be flat or in the form of dashes or dots. An adhesive bond may be formed, for example, by applying an adhesive or by means of an adhesive tape. A welding bond may be formed, for example, by heat or by ultrasound.

In a preferred embodiment, the cover layer extends laterally beyond the wound contact layer, and at least one section of the cover layer that extends laterally beyond the wound contact layer is in adhesive form. This allows particularly stable securing of the wound dressing to the skin around the wound to be achieved. Because the cover layer extends laterally beyond the wound contact layer, the cover layer, when the wound dressing is applied properly, comes into contact solely with the skin around the wound, not with the wound bed. Accordingly, strong adhesives can also be used in the section that protrudes beyond the wound contact layer, which should not come into direct contact with the wound bed. In an alternative embodiment, the cover layer extends in lateral direction at most to an edge of the wound contact layer, but not beyond the edge. When the wound dressing is applied properly, it is solely the wound contact layer that is in direct contact with the tissue, not the cover layer.

Preferably, the connection opening has an assigned air-permeable and liquid-impermeable filter unit that has a filtering effect between the receiving space and the environment. Because the filter unit is designed to be air-permeable, the filter unit can communicate a negative pressure into the receiving space. Because the filter unit is liquid-impermeable, the filter unit prevents wound exudate from escaping from the wound dressing when the wound dressing is used in negative pressure therapy. Wound exudate escaping from the wound dressing could otherwise contaminate and/or damage the downstream negative pressure source. The filter unit is preferably disposed on a side of the cover layer facing the wound contact layer, i.e. in the receiving space.

The wound contact layer preferably comprises a perforated film. In a perforated film, a multitude of passage openings are formed in the film, for example by punching. Accordingly, wound exudate can be distributed along the wound contact layer into the receiving space. The perforated film is preferably a perforated polymer film.

In a preferred embodiment, the wound contact layer comprises a silicone or a hydrogel. A silicone or hydrogel is particularly suitable for the formation of the wound contact layer. This is because silicones and hydrogels typically adhere sufficiently strongly to dry tissue, for example the skin around the wound, for reliable securing of the wound dressing. On the other hand, silicones and hydrogels adhere slightly at most to moist or wet tissue, for example the wound bed, such that sticking to the wound bed is avoided. More preferably, the wound contact layer comprises a perforated film, in particular perforated polymer film, which is coated with a silicone or a hydrogel on its side facing away from the cover layer.

The wound dressing is preferably packaged as a ready-made wound dressing in an outer package. A ready-made wound dressing is a wound dressing in which the individual layers are already bonded to each other as intended during storage. Such a ready-made wound dressing is straightforward to use.

The object to be achieved is also achieved by a negative pressure wound therapy kit comprising a wound dressing having the features described above. The negative pressure wound therapy kit according to the invention also comprises a connecting device comprising an elongated single-lumen or multilumen connecting tube comprising a distal end section and a proximal end section, where the distal end section is fluidically connectable to the connection opening in a negative pressure-tight manner, and wherein the proximal end section is fluidically connectable to a negative pressure source in a negative pressure-tight manner.

The terms “distal” and “proximal” describe the arrangement relative to the negative pressure source. A proximal section of an element is situated closer to the negative pressure source than a distal section of the same element. In this respect, for example, the proximal end section of the connecting tube, when the connecting tube is used as intended, is closer to the negative pressure source than the distal end section.

In respect of the advantages achievable with the negative pressure wound therapy kit, reference is made to the statements in this regard relating to the wound dressing. Further configuration of the negative pressure wound therapy kit can be accomplished using the features described in association with the wound dressing.

The object to be achieved is also achieved by a negative pressure wound therapy system which comprises a negative pressure wound therapy kit having the features described above. The negative pressure wound therapy system according to the invention furthermore comprises a negative pressure source, wherein the proximal end section of the connecting tube is fluidically connectable to the negative pressure source in a negative pressure-tight manner.

In respect of the advantages achievable with the negative pressure wound therapy system, reference is made to the statements in this regard relating to the wound dressing. Further configuration of the negative pressure wound therapy system can be accomplished using the features described in association with the wound dressing.

The invention is described in detail hereinafter with reference to the figures, where elements that are the same or functionally the same may be given reference symbols only in one instance. The figures serve as an example and should not be regarded as limiting. The figures show:

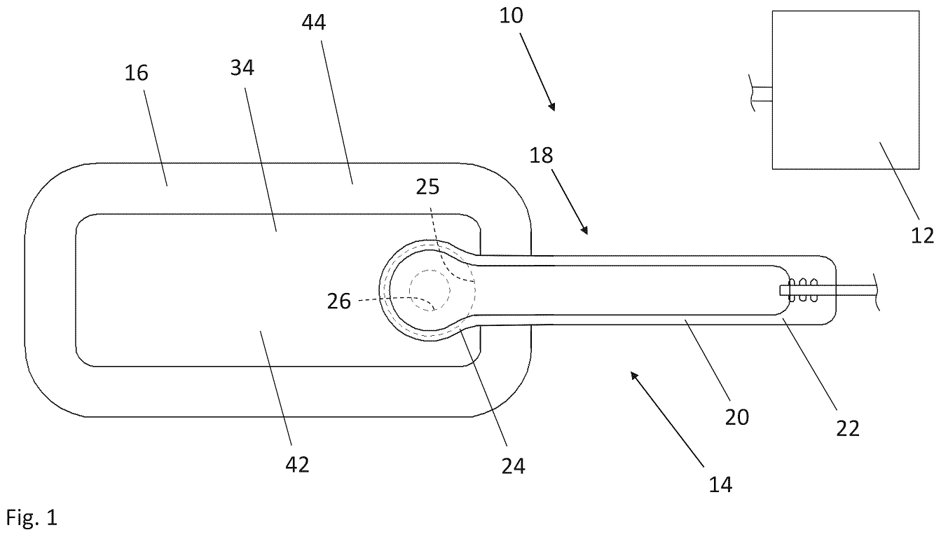

FIG. 1 a negative pressure wound therapy system comprising a wound dressing,

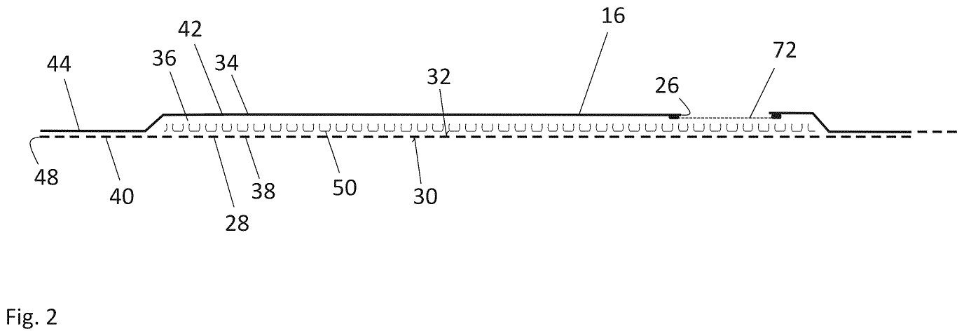

FIG. 2 a section diagram of a first working example of the wound dressing,

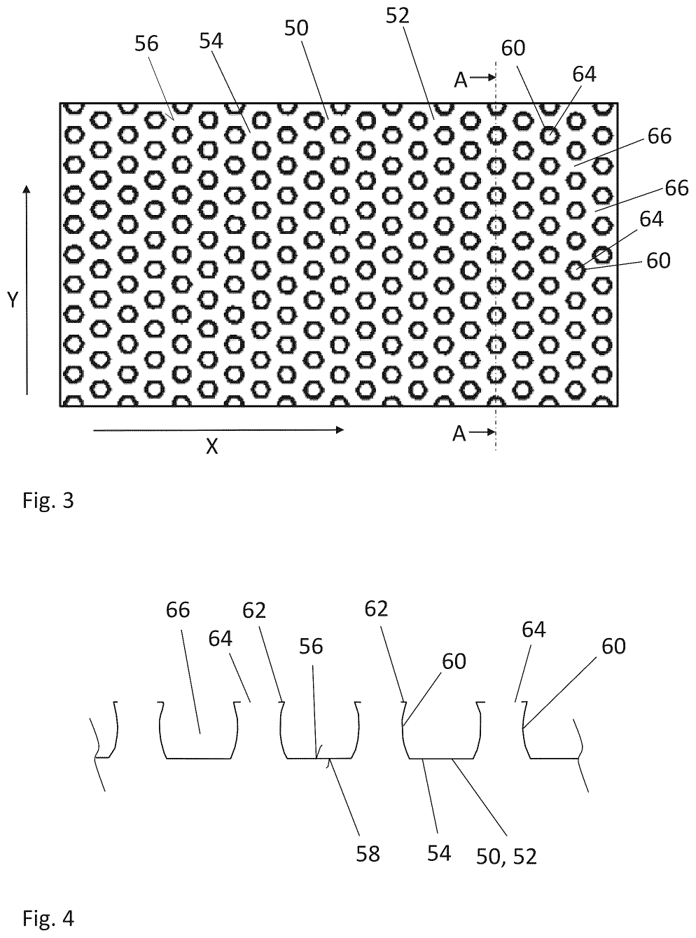

FIG. 3 a top view of a distributor layer of the wound dressing,

FIG. 4 a section diagram of the distributor layer shown in FIG. 3 along the section line A-A,

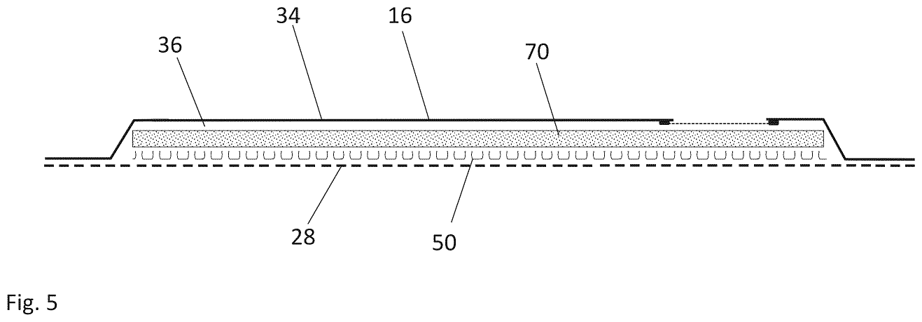

FIG. 5 a section diagram of a second working example of the wound dressing,

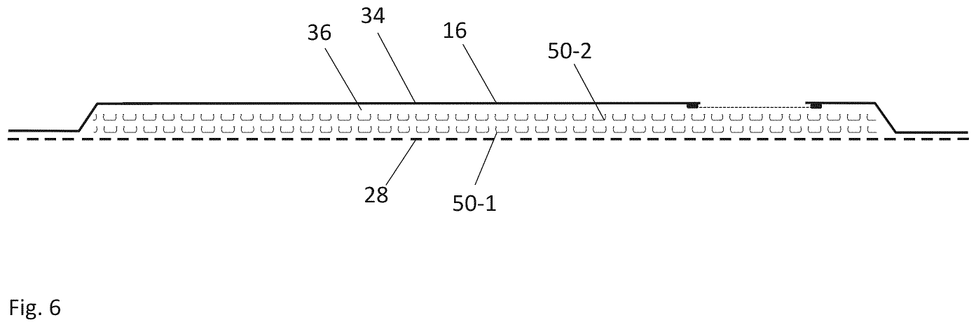

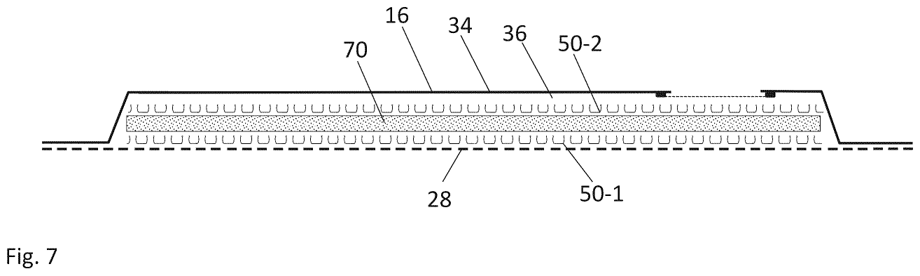

FIG. 6 a section diagram of a third working example of the wound dressing,

FIG. 7 a section diagram of a fourth working example of the wound dressing and

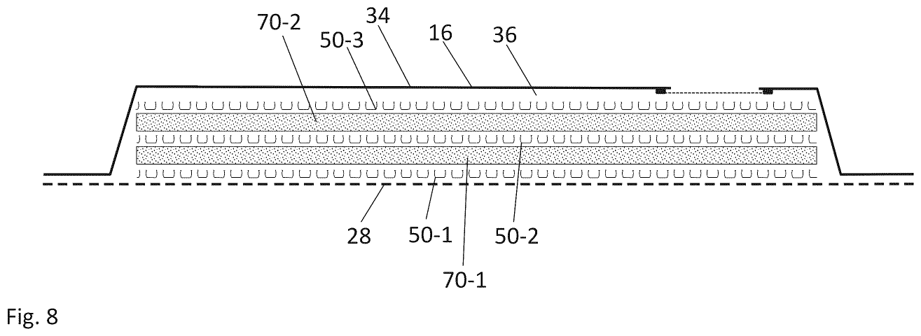

FIG. 8 a Section Diagram of a Fifth Working Example of the Wound Dressing.

FIG. 1 shows a negative pressure wound therapy system 10 for use in negative pressure wound therapy. The negative pressure wound therapy system 10 comprises a negative pressure source 12 and a negative pressure wound therapy kit 14. The negative pressure wound therapy kit 14 comprises a wound dressing 16 and a connecting device 18. The connecting device 18 comprises an elongate single-lumen or multilumen connecting tube 20 with a proximal end section 22 and a distal end section 24. The proximal end section 22 is fluidically connectable to the negative pressure source 12 in a negative pressure-tight manner. The distal end section 24 is fluidically connectable to a connection opening 26 in the wound dressing 16 in a negative pressure-tight manner. For this purpose, the distal end section 24 comprises a fluid inlet 25. In the negative pressure wound therapy system 10 shown in FIG. 1, the distal end section 24 is already connected to the connection opening 26, and so the connection opening 26 is covered by the distal end section 24.

There follows a detailed elucidation of the construction of the wound dressing 16 with additional reference to FIG. 2. FIG. 2 shows a schematic section diagram of a first working example of the wound dressing 16. It should be pointed out that layers of the wound dressing 16 that are stacked directly one on top of another are shown spaced apart from one another in FIG. 2 for better comprehensibility. By contrast with this illustration, when the wound dressing 16 is used, layers of the wound dressing 16 that are stacked directly one on top of another are in direct contact.

The wound dressing 16 comprises a wound contact layer 28. The wound contact layer 28 comprises a smooth first side 30 for application to the wound bed of a wound and a smooth second side 32 facing away from the first side 30.

The wound dressing 16 also comprises an air-impermeable cover layer 34 for airtight sealing of the wound. The connection opening 26 that has already been mentioned above is formed in the cover layer 34. The cover layer 34 preferably comprises a polymer film, especially a polyurethane film. A receiving space 36 is formed between the wound contact layer 28 and the cover layer 34.

The wound contact layer 28 is designed to be fluid-permeable for draining of wound exudate from the wound into the receiving space 36. In the present case, the wound contact layer 28 comprises a polymer film which is coated on its wound-facing side with a silicone or with a hydrogel. The wound contact layer 28, i.e. the polymer film together with the silicone or the hydrogel, is perforated. In this respect, the wound contact layer 28 comprises a multitude of perforations or passage openings that are distributed in the wound contact layer 28. The perforations can be used to drain wound exudate from the wound cavity into the receiving space 36.

The wound contact layer 28 comprises a central region 38 and an edge region 40 surrounding the central region 38. The cover layer 34 comprises a central region 42 and an edge region 44 enclosing the central region 42. In the present case, the edge region 40 of the wound contact layer 28 is bonded circumferentially to the edge region 44 of the cover layer 34. The receiving space 36 is accordingly formed solely between the central region 38 of the wound contact layer 28 and the central region 42 of the cover layer 34.

Preferably, the edge areas 40 and 44 are bonded to one another by an adhesive bond. More preferably for this purpose, a side of the cover layer 34 that faces the wound contact layer 28 is coated at least in the edge region 44 with an adhesive, for example with an acrylate adhesive. Alternatively, the edge areas 40 and 44 are bonded to one another by a weld bond.

The bonding of the edge regions 40 and 44 to one another has the effect that further layers disposed in the receiving space 36 are held securely in the receiving space 36. Accordingly, slight slippage of these layers at most is possible. This results in the advantage that it is possible to dispense with bonding of the layers disposed in the receiving space 36 to one another and with bonding of the layers to the wound contact layer 28 and/or to the cover layer 34.

When the wound dressing 16 is applied to the wound, with the distal end section 24 fluidically connected to the connection opening 26 in a negative pressure-tight manner and the proximal end section 22 fluidically connected to the negative pressure source 12 in a negative pressure-tight manner, a negative pressure can be established by the negative pressure source 12 between the cover layer 34 and the wound in order to effectively drain wound exudate from the wound.

The edge region 44 of the cover layer 34, in the embodiments shown in the figures, projects at most up to an edge 48 of the wound contact layer 28, but not beyond the edge 48. The cover layer 34 accordingly does not come into contact with the skin around the wound when the dressing 16 is applied properly. In a further working example, the edge region 44 of the cover layer 34 extends laterally at least in sections beyond the edge 48 of the wound contact layer 28. This has the advantage that particularly reliable securing of the wound dressing 16 to the tissue can be achieved. The aforementioned adhesive on the side of the cover layer 34 facing the wound contact layer 28 then comes into contact with the skin around the wound. Such a lateral protrusion of the cover layer 34 is indicated by dotted lines in FIG. 2.

The wound dressing 16 also comprises an air-permeable and liquid-impermeable filter unit 72. The filter unit 72 is assigned to the connection opening 26 and has a filtering effect between the receiving space 36 and the environment. In the present case, the filter unit 72 is disposed on the side of the cover layer 34 facing the wound contact layer 28.

In the first working example of the wound dressing 16 shown in FIG. 2, a distributor layer 50 is disposed in the receiving space 36. There follows a detailed elucidation, with additional reference to FIGS. 3 and 4, of the design of distributor layer 50. In this regard, FIG. 3 shows a top view of the distributor layer 50. FIG. 4 shows a section diagram of the distributor layer 50 along the section line A-A shown in FIG. 3.

The distributor layer 50 is extended and formed in two spatial directions X and Y in order to distribute wound exudate in the spatial directions X and Y. In addition, the distributor layer 50 promotes distribution of the negative pressure established in the wound cavity. The distributor layer 50 is formed by a flat material web section 52. The flat material web section 52 comprises a plane 54 having a first side 56 and a second side 58 facing away from the first side 56.

The first side 56 of the flat material web section 52 is structured by elevations 60 integrally with the plane 54. In at least some of the elevations 60, an end-face passage opening 64 is formed in the raised end faces 62 of the elevations 60 in question. In the present case, an end-face passage opening 64 is formed in each of the elevations 60. A contiguous interspace 66 is formed between the elevations 60 such that wound exudate is distributable in the spatial directions X and Y in the interspace 66. The second side 58 of the flat material web section 52 that faces away from the structured first side 56 is smooth.

The flat material web section 52 on the first side 56 preferably has an elevation density of at least 200 elevations per cm2 to at most 400 elevations 60 per cm2. In the present case, the elevation density is 280 elevations 60 per cm2. The diameter of the end-face passage openings 64 is preferably at least 200 μm and at most 500 μm.

For formation of the contiguous interspace 66, the elevations 60 are isolated from one another in insular form. In the working example shown, the shape of the elevations 60 resembles a single-sheet hyperboloid. In the present case, the elevations 60 are thus hyperboloid. For this purpose, an outer wall of the elevations 60 has anticlastic curvature. Alternatively, the elevations 60 are preferably conical, i.e. frustoconical, cylindrical or frustopyramidal.

The elevations 60 are preferably arranged in such a distribution that, at least in a central region of the flat material web section 52, each of the elevations 60 is surrounded by three to eight further elevations 60, where the further elevations 60 together form a regular polygon. In the present case, the further elevations 60 collectively form a regular hexagon. At least in the central region of the flat material web section 52, each of the elevations 60 is thus surrounded by six other elevations 60. Such an arrangement of the elevations 60 leads to a particularly uniform distribution of wound exudate in spatial directions X and Y.

The flat material web section 52 in the present case is a polymer film 52. The polymer film 52 is preferably made of polyethylene, polyurethane, polyvinylchloride or a mixture thereof. These plastics have the advantage that, on the one hand, a flexibly deformable distributor layer 50 can be obtained. This results in a wound dressing 16 having advantageous drapability. Moreover, the aforementioned plastics also have adequate rigidity. In this respect, the plastics ensure that the contiguous interspace 66 is maintained with use of the wound dressing 16 in spite of the negative pressure established.

The elevations 60 are made by vacuum thermoforming. Preferably, for this purpose, the still-molten extruded or calendered flat material web, in particular polymer film web, is passed across a rotating perforated roll under reduced pressure. The flat material web is then pulled region by region into holes of the perforated roll under reduced pressure by a negative pressure in the perforated roll under reduced pressure, which forms the elevations 60. The shape of the obtained elevations 60 is determined here, for example, by the outline of the holes and by the negative pressure used. Preferably, the negative pressure in the perforated roll under reduced pressure is adjusted such that the end faces 62 of the elevations 60 open up. The elevations 60 and the passage openings 64 are thus formed in the same process step.

The distributor layer 50 is disposed in the receiving space 36 such that the elevations 60 protrude in the direction of the cover layer 34. This supports the transport of wound exudate through the distributor layer 50 in the direction of the cover layer 34. Specifically, capillary forces that act on the wound exudate in the elevations 60 promote transport in the direction of the cover layer 34.

FIGS. 5 to 8 shows schematics of various further embodiments of the wound dressing 16. The working examples disclosed in FIGS. 5 to 8 differ from the working example shown in FIG. 2 with regard to the layers disposed in the receiving space 36. With regard to the formation of the wound contact layer 28, the cover layer 34 and existing distributor layers 50, reference is made to the above details of these layers. It is also the case that the wound dressings 16 shown in FIGS. 5 to 8 may take the form of a wound dressing in the negative pressure wound therapy kit 14 or in the negative pressure wound therapy system 10.

FIG. 5 shows a section diagram of a second working example of the wound dressing 16. In the wound dressing 16 shown in FIG. 5, a distributor layer 50 and an absorption layer 70 are disposed in the receiving space 36.

The absorption layer 70 is designed to absorb wound exudate and store absorbed wound exudate. Various materials are conceivable for formation of the absorption layer 70, in particular hydrophilic materials. The absorption layer 70 preferably comprises superabsorbent polymers, superabsorbent fibers, cellulose and/or a hydrophilic nonwoven material.

The distributor layer 50 and the absorption layer 70 are stacked directly one on top of the other in the receiving space 36, such that the distributor layer 50 is in direct contact with the absorption layer 70. The distributor layer 50 is disposed between the absorption layer 70 and the wound contact layer 28. The elevations 60 of the distributor layer 50 face the absorption layer 70. The lateral distribution of wound exudate in the interspace 66 achieves the effect that a large portion of the surface area of the absorption layer 70 is used for the absorption of wound exudate. This prevents blockage of the absorption layer 70. Such a blockage would be more likely if the wound exudate were only to be absorbed by the absorption layer 70 at individual isolated sites.

FIG. 6 shows a section diagram of a third working example of the wound dressing 16. In the wound dressing 16 shown in FIG. 5, a first distributor layer 50-1 and a second distributor layer 50-2 are disposed in the receiving space 36.

The first distributor layer 50-1 and the second distributor layer 50-2 are stacked one on top of the other in the receiving space 36 such that the elevations 60 of the distributor layers 50-1 and 50-2 protrude in the same direction, in the present case in the direction of the cover layer 34. This means that the capillary forces act in the direction of the cover layer 34 both in the elevations 60 of the first distributor layer 50-1 and in the elevations 60 of the second distributor layer 50-2. The providing of two distributor layers 50-1 and 50-2 can increase the capacity of the wound dressing 16 for wound exudate.

FIG. 7 shows a section diagram of a fourth working example of the wound dressing 16. In the wound dressing 16 shown in FIG. 7, a first distributor layer 50-1, a second distributor layer 50-2 and an absorption layer 70 are disposed in the receiving space 36.

The distributor layers 50-1 and 50-2 are disposed on opposite sides of the absorption layer 70. The first distributor layer 50-1 is disposed between the wound contact layer 28 and the absorption layer 70. The second distributor layer 50-2 is disposed between the cover layer 34 and the absorption layer 70. The distributor layers 50-1 and 50-2 are arranged such that the elevations 60 protrude in the same direction, in the present case in the direction of the cover layer 34. The second distributor layer 50-2 prevents individual regions of the absorption layer 70 from being isolated from the negative pressure. Such isolation of individual regions could impair the efficiency of the wound dressing 16 with regard to the draining of wound exudate from the wound.

FIG. 8 shows a section diagram of a fifth working example of the wound dressing 16. In the wound dressing 16 shown in FIG. 8, a first distributor layer 50-1, a second distributor layer 50-2, a third distributor layer 50-3, a first absorption layer 70-1 and a second absorption layer 70-2 are disposed in the receiving space.

The second distributor layer 50-2 is disposed between the first absorption layer 70-1 and the second absorption layer 70-2. The first distributor layer 50-1 is disposed on a side of the first absorption layer 70-1 that faces away from the second absorption layer 70-2. The third distributor layer 50-3 is disposed on a side of the second absorption layer 70-2 that faces away from the first absorption layer 70-1. The distributor layers 50-1, 50-2 and 50-3 are disposed in the receiving space 36 such that their elevations 60 protrude in the same direction, in the present case in the direction of the cover layer 34.

1-23. (canceled)

24. A wound dressing for use in negative pressure wound therapy, comprising:
a wound contact layer for application to a wound bed of a wound, where the wound contact layer comprises a smooth first side and a smooth second side facing away from the first side,
an airtight cover layer for airtight closure of the wound, where a receiving space is formed between the cover layer and the wound contact layer, where the wound contact layer is in liquid-permeable form for discharge of wound exudate from the wound into the receiving space, and where a connection opening for negative pressure-tight fluid connection of the receiving space to a negative pressure source is formed in the cover layer, and
at least one distributor layer disposed in the receiving space, wherein the distributor layer extends in two spatial directions (X, Y) and is able to distribute wound exudate in the spatial directions (X, Y), wherein the distributor layer is formed by a flat material web section, wherein a first side of the flat material web section is structured by elevations integrally with a plane of the flat material web section, wherein an end-face passage opening is formed in at least some of the elevations, and wherein a contiguous interspace is formed between the elevations such that wound exudate is distributable in the spatial directions (X, Y) in the interspace.

25. The wound dressing of claim 24, wherein the elevations are conical, cylindrical, frustopyramidal or hyperboloid.

26. The wound dressing of claim 24, wherein at least in a central region of the flat material web section, each of the elevations is surrounded by three to eight further elevations, where the further elevations together form a regular polygon.

27. The wound dressing of claim 24, wherein a second side of the flat material web section facing away from the structured first side is smooth.

28. The wound dressing of claim 24, wherein the flat material web section is made from polyethylene, polyurethane, polyvinylchloride or mixtures thereof.

29. The wound dressing of claim 24, wherein the distributor layer is disposed in the receiving space such that the elevations protrude in the direction of the cover layer.

30. The wound dressing of claim 24, wherein at least one further distributor layer disposed in the receiving space extends in two spatial directions (X, Y) and is able to distribute wound exudate in the spatial directions (X, Y), wherein the further distributor layer is formed by a flat material web section, in that a first side of the flat material web section is structured by elevations integrally with a plane of the flat material web section, wherein an end-face passage opening is formed in at least some of the elevations, wherein a contiguous interspace is formed between the elevations such that wound exudate is distributable in the spatial directions (X, Y) in the interspace, and wherein the distributor layer and the further distributor layer are stacked one on top of the other.

31. The wound dressing of claim 30, wherein the distributor layer and the further distributor layer are arranged such that the elevations of the distributor layers protrude in the same direction.

32. The wound dressing of claim 24, wherein at least one absorption layer disposed in the receiving space is able to absorb wound exudate and store absorbed wound exudate.

33. The wound dressing of claim 32, wherein the distributor layer or at least one of the distributor layers is disposed between the absorption layer and the wound contact layer.

34. The wound dressing of claim 32, wherein the distributor layer or at least one of the distributor layers is disposed between the absorption layer and the cover layer.

35. The wound dressing of claim 32, wherein the absorption layer is in direct contact with the distributor layer and/or the further distributor layer.

36. The wound dressing of claim 32, wherein at least one further absorption layer disposed in the receiving space is able to absorb wound exudate and store absorbed wound exudate, and wherein the absorption layer and the further absorption layer are stacked one on top of the other.

37. The wound dressing of claim 36, wherein the distributor layer or at least one of the distributor layers is disposed between the absorption layer and the further absorption layer.

38. The wound dressing of claim 24, wherein the cover layer comprises a central region and an edge region enclosing the central region, wherein the wound contact layer comprises a wound contact layer central region and a wound contact layer edge region enclosing the wound contact layer central region, and wherein the edge region of the cover layer is bonded to the edge region of the wound contact layer such that the receiving space is formed solely between the central region of the cover layer and the central region of the wound contact layer.

39. The wound dressing of claim 24, wherein the cover layer extends laterally beyond the wound contact layer, and in that at least one section of the cover layer that extends laterally beyond the wound contact layer is in adhesive form.

40. The wound dressing of claim 24, wherein the connection opening has an assigned air-permeable and fluid-impermeable filter unit that has a filtering effect between the receiving space and the environment.

41. The wound dressing of claim 24, wherein the wound dressing is packaged as a ready-made wound dressing in an outer package.

42. A negative pressure wound therapy kit comprising a wound dressing as claimed in claim 24 and comprising a connecting device comprising an elongated single-lumen or multilumen connecting tube with a distal end section and a proximal end section, wherein the distal end section is fluidically connectable to the connection opening in a negative pressure-tight manner, and wherein the proximal end section is fluidically connectable to a negative pressure source in a negative pressure-tight manner.

43. A negative pressure wound therapy system comprising the negative pressure wound therapy kit as claimed in claim 42 and comprising a negative pressure source, wherein the proximal end section of the connecting tube is fluidically connectable to the negative pressure source in a negative pressure-tight manner.
